(19) United States
(12) Patent Application Publication
(10) Pub. No.: US 2008/0312147 A1
(43) Pub. Date: Dec. 18, 2008

(54) Compositions and Methods for the Regulation of Hair Growth

(75) Inventor: Nazli Azimi, San Juan Capistrano, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: NUGENE INC.

(21) Appl. No.: 11/764,040

(22) Filed: Jun. 15, 2007

(51) Int. Cl. A61K 38/18; A61K 38/06; A61K 38/07; A61P 17/14; A61K 38/08; A61K 38/10

(52) U.S. Cl. 514/12; 514/13; 514/14; 514/15; 514/16; 514/17; 514/18

(57) ABSTRACT

A novel hair loss prevention and hair restoration composition, is applied topically to the scalp. The composition and methods of application stimulate the hair follicle, encourage angiogenesis, and improve blood circulation throughout the area where the composition is applied. Moreover, formation of di-hydroxytestosterone is inhibited using copper peptides that interfere with 5-α-reductase activity that would otherwise convert testosterone to di-hydroxytestosterone.

### BACKGROUND

The present disclosure relates to the mitigation or prevention of hair loss and restoration of hair growth.

### SUMMARY

A novel hair loss prevention and hair restoration composition is applied topically to the scalp. The composition and methods of application stimulate the hair follicle, encourage angiogenesis, and improve blood circulation throughout the area where the composition is applied. Moreover, formation of di-hydroxytestosterone is inhibited using copper peptides that interfere with 5-α-reductase activity that would otherwise convert testosterone to di-hydroxytestosterone.

According to a feature of the present disclosure, a method is disclosed comprising, in combination: (1) providing a composition having an agent that interferes with 5-α-reductase to prevent hair loss; and (2) providing at least one of a hair follicle stimulator, a scalp blood supply circulation stimulator, and an agent promoting angiogenesis near hair follicles, to stimulate hair growth.

According to a feature of the present disclosure, a composition is disclosed comprising an agent that interferes with 5-α-reductase, and at least one of a hair follicle stimulator, a scalp blood supply circulation stimulator, and an agent promoting angiogenesis near hair follicles.

According to a feature of the present disclosure, a composition is disclosed comprising a 5-α-reductase interfering agent, the agent having at least a peptide sequence comprising copper.

### DRAWINGS

The above-mentioned features and objects of the present disclosure will become more apparent with reference to the following description taken in conjunction with the accompanying drawings wherein like reference numerals denote like elements and in which:

FIG. 1A is a block diagram illustrating an embodiment of the mechanism by which di-hydroxytestosterone (DHT) causes hair loss;

FIG. 1B is a block diagram illustrating an embodiment of a mechanism for preventing the formation of DHT, and therefore preventing hair loss;

FIG. 1C is a block diagram illustrating an embodiment of a hair loss prevention method;

FIG. 2 is a block diagram illustrating an embodiment of a hair growth stimulation method;

FIG. 3 is a cross sectional view of an embodiment of a nanosome having at least one agent for hair loss prevention or hair growth; and

FIG. 4 is a flow diagram of a method an embodiment of delivering an embodiment of a composition disclosed in the present disclosure via nanosomes.

### DETAILED DESCRIPTION

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, biological, electrical, functional, and other changes may be made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction and shall not indicate an exclusive disjunction unless expressly indicated as such or notated as “xor.”

The inventor of the present disclosure has invented a baldness treatment and a hair loss prevention method. The methods and compositions address prevention of hair loss as well as stimulation of new hair growth. Thus, the methods and compositions of the present disclosure are appropriate for patients who have already experienced hair loss, and for patients who are predisposed to hair loss or in the early stages of hair loss.

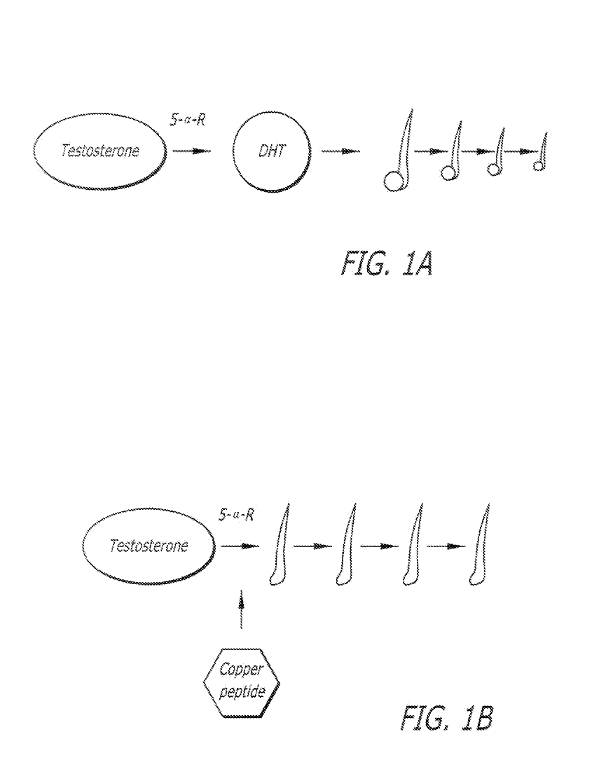

According to an embodiment shown in FIG. 1A, hair loss is induced by the formation of di-hydroxytestosterone (DHT) from the hormone testosterone. The formation of DHT is mediated, at least in part, by 5-α-reductase, which converts testosterone into DHT. DHT binds to androgen receptors in hair follicles, triggering an autoimmune response that causes miniaturization and shrinking of the hair follicles. Over time, the shrinking of the hair follicles results in a shortening of the growth cycle for the hair follicles until no further growth occurs.

Inhibition of 5-α-reductase prevents DHT formation. Thus, without the presence of DHT, the growth cycles of the hair follicles will not be interrupted and hair loss will not occur. Therefore, by inhibiting 5-α-reductase, hair loss is prevented.

According to embodiments, short, copper containing peptides have been shown to interrupt the activity of 5-α-reductase. According to embodiments, copper, for example Cu(II), is coupled to a peptide. The copper containing peptide interrupts the activity of 5-α-reductase by preventing binding of testosterone to the active site of the 5-α-reductase. For example, a peptide having the amino acid sequence Gly-His-Lys-Cu(II) has been shown to interrupt the activity of 5-α-reductase. Other peptide sequences that have been shown to interrupt 5-α-reductase include Cu(II)-His-Val-His and His-Ala-His-Cu(II).

According to embodiments illustrated in FIG. 1C, a method for preventing hair loss is illustrated. By interfering with 5-α-reductase 102, synthesis of DHT is prevented 104. Consequently, hair loss is prevented 106.

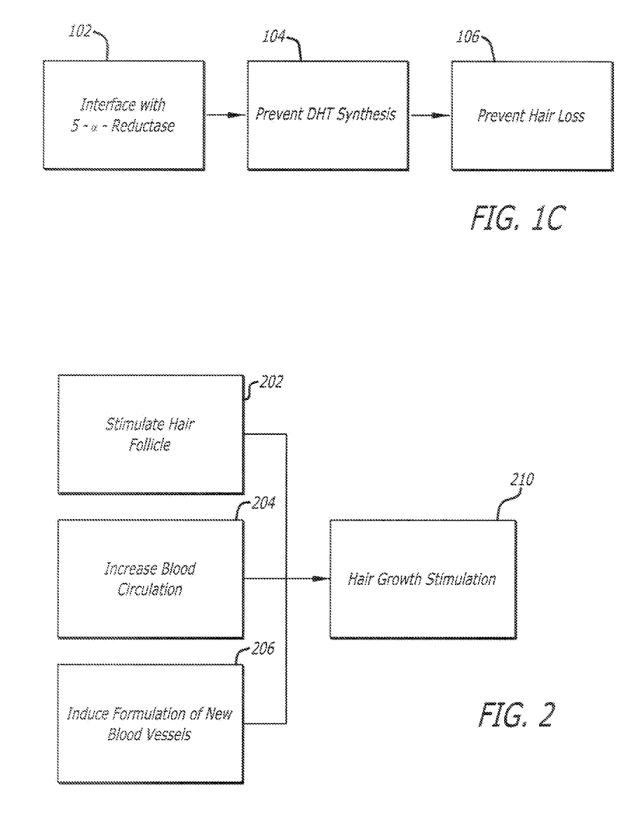

As illustrated in FIG. 2 and according to embodiments, in addition to a hair loss prevention, the present disclosure contemplates renewed hair growth for areas where hair has stopped growing. According to embodiments, the hair growth regimen is three pronged: the hair follicle is stimulated 202, blood circulation near the follicle is increased 204, and formation of new blood vessels and strengthen the extracellular matrix is induced 206. The combination these agents has been shown to stimulate new hair growth 210.

As the hair follicle growth cycle ceases due to the activity of DHT, the blood supply to the hair follicle is decreased. Consequently, as new hair growth is induced, the blood supply must be increased to carry to the burgeoning hair follicle oxygen and nutrients. Additionally, the extracellular matrix must be strengthened, which promotes normal hair growth cycle. Increasing fibroblast density in the matrix causes hair thickness therefore to be increased.

According to embodiments, hair follicle stimulation is accomplished by adding the growth factors to stimulate the hair follicle, increase angiogenesis and near the follicle, and strengthen the extracellular matrix to the location where hair growth is desired. For example and according to embodiments, insulin-like growth factor-1 (IGF-1) is known to stimulate hair follicles and generally strengthen the extracellular matrix.

According to embodiments, blood circulation is increased by adding a fibroblast growth factor (FGF), such as basic fibroblast growth factor (bFGF), Fibroblast growth factors have been shown to induce angiogenesis in endothelial cells. Furthermore, FGFs tend to cause the proliferation of and encourage health of endothelial cells. In addition, fibroblast growth factors stimulate growth of fibroblasts. Fibroblasts form a core cell type in the papilla of the hair follicle. In balding areas, the papilla tends to decrease in size. Thus, by encouraging fibroblast growth, the papilla of the follicle grows rather than shrinks, inducing renewed hair growth and thickness.

Likewise, for the hair follicle to correctly function, blood vessel formation and increased blood circulation is required to provide nutrients to the hair follicle, as well as increased oxygen. Artisans will note that FGFs stimulate angiogenesis. When coupled with vascular endothelial growth factor (VEGF), blood vessel formation and interconnectedness is enhanced. Indeed, research has shown that the combination of VEGF and FGFs synergistically induces blood vessel formation and increased circulation. Naturally, as blood vessels form and circulatory networks are created in an area of balding, increased oxygen and nutrients become available to encourage and aid hair function resulting in new or increased hair growth.

Therefore, a cocktail of growth factors are simultaneously applied to the areas where hair growth is desired to simultaneously restore hair growth cycle, induce the follicle's hair growth functions, and provide additional nutrient and oxygen supply. Together, these growth factors have the cumulative effect to effect hair growth.

According to embodiments, other growth factors are added to further enhance the growth of hair. Examples of other growth factors that may be used by themselves or in combination with one or more of the others include: keratinocyte growth factor (KGF), a growth factor inducing growth and health of keratinocytes, which comprise around 90% of epidermal cells; transforming growth factor β-3 (TGF-β3), which promotes the growth and development of the extracellular matrix around epidermal and dermal cells; and thioredoxin (TRX), an antioxidant.

To effectively deliver growth factors according to the methods for treating hair loss, they must be effectively absorbed through into the skin without degradation. According to embodiments, nanosomes may be used to deliver the growth factors to their target locations.

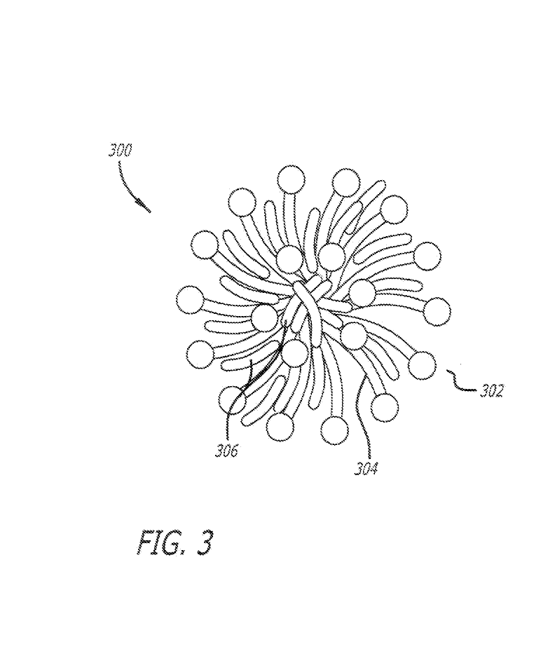

According to embodiments shown in FIG. 3, nanosomes 300, which are essentially smaller (−30 nm) liposomes, are formed from a layer of molecules having hydrophobic head 302 and hydrophilic tail 304. For example, a nanosome may be constructed having a phospholipid and cholesterol bilayer. Like cells, the phospholipids aggregate together to form a “capsule” with the hydrophobic portion of the bilayer on the outside and the hydrophilic portion on the inside, much as oil micelles form in water. Encapsulated in nanosome 300 are one or more agents 306 to be delivered.

Nanosomes may be desirable, according to embodiments, because they improve bioavailability of the agents encapsulated within them. Bioavailability is increased because more agent is actually delivered to its target. Moreover, nanosomes readily cross through the gastrointestinal membrane, thereby effectively transporting the agents into the blood stream. Additionally, because the bioavailability is increased, less agent must be used, which reduces the costs associated with producing and delivering agents to effect a result. Because the agents encapsulated in the nanosome are protected from the environment, the agents have a longer shelf life and are more stable on average than those not encapsulated. Importantly, agents encapsulated within a nanosome can undergo pasteurization without destroying the agent in the process.

Most importantly, however, the nanosome protects the agents from oxidation, hydrolysis, reduction, and enzymatic reaction, thereby allowing the agent to arrive at its target in an active state. Finally, nanosome encapsulation masks odor and taste of the agents, making them more pleasing to those who use the agents.

The growth factors and hair prevention agents of the present disclosure may be encapsulated in nanosomes, and the nanosomes put into a delivery vehicle. Thus, they will be effectively delivered through the skin or into the blood stream. Thereafter, the nanosomes may be absorbed into cells by fusing with cellular membranes or via endocytosis, for example.

Naturally, because the external surface of the nanosome is hydrophobic, nanosomes are fat soluble and excellent tool for delivering pharmacological agents through the skin or the digestive tract directly to the cell without exposing the active agents to degradation events prior to delivery. Additionally, nanosomes allow agents that would otherwise only inefficiently arrive at the target cells, for example, water soluble agent applied topically. Thus, in combination, with the novel agents disclosed herein, nanosomes provide a delivery device to efficiently cause the agents to be delivered intracellulary.

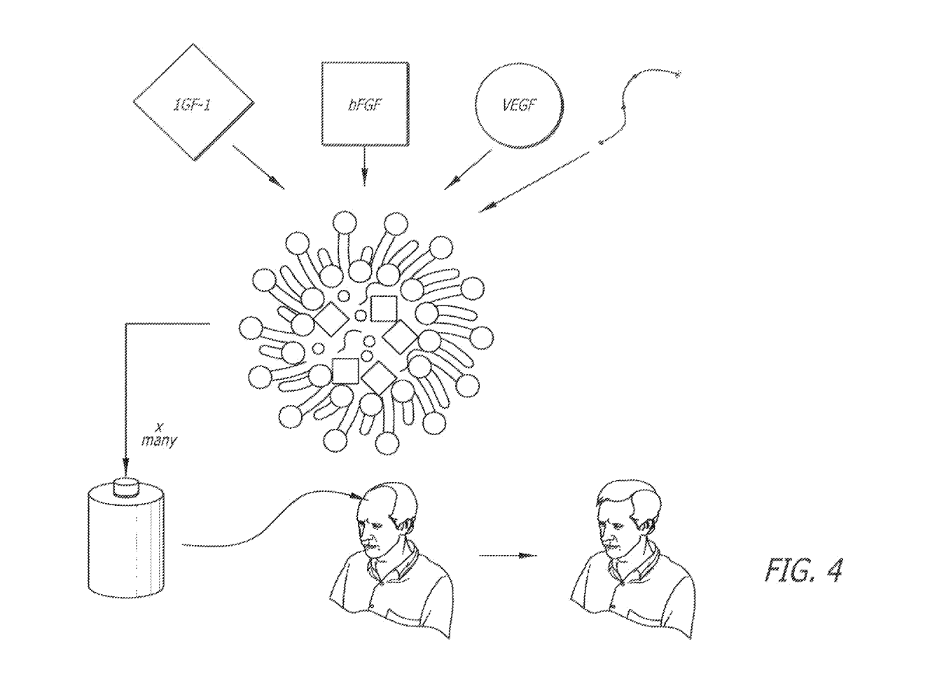

According to embodiments and as illustrated in FIG. 4, the hair loss prevention or hair growth agents are encapsulated in nanosome 300 and disposed in a variety of delivery vehicles 402. For example, the copper peptides 306d and growth factors, such as IGF-1 306a, bFGF 306b, and VEGF 306c are encapsulated into nanosomes, and nanosomes 300 put into delivery vehicles 402, such as hair shampoos, hair conditioners, hair sprays, massage oils, and topic serums for direct delivery to the areas affected by or predisposed to hair loss. Indeed, because nanosomes 300 are stable in a water based solutions, nearly any such solution that can be applied to hair loss areas forms an effective delivery mechanism for nanosomes having the hair loss prevention or hair growth agents. Delivery vehicle 402 is applied in process 410 to balding area 412A to effect growth of new hair 412B. Likewise, delivery vehicle 402 may be pills, beverages, and other ingestibles for delivery of nanosomes 300 encapsulating hair loss prevention and hair growth agents into the blood stream.

### EXAMPLE 1

According to embodiments, a hair loss prevention composition contains an agent that interferes with 5-α-reductase. For example, as disclosed herein, the agent may be a peptide having copper. Such a peptide may be disposed with in a nanosome in a concentration of 1-10000 μg/ml. According to embodiments, concentrations as low as 1-5 μg/ml are effective for preventing hair loss.

According to embodiments, the peptide having the copper may comprise at least Gly-His-Lys-Cu(II) (GHK-Cu(II)). According to other embodiments, other three amino acid coupled to copper peptides may be used instead of or in addition to GHK-Cu(II). Moreover, longer peptide sequences having at least one copper molecule are also effective and may be used instead of or in addition to the three amino acid coupled to copper peptides.

### EXAMPLE 2

According to embodiments, a hair loss prevention and hair growth composition contains both an agent that interferes with 5-α-reductase and growth factors to stimulate hair regrowth. The 5-α-reductase interfering agent may be as disclosed in EXAMPLE 1, according to embodiments. Artisans will readily recognize that the hair growth factors may be used or packaged separately from the hair loss prevention agents. However, artisans will also recognize the desirability of both inhibit further hair loss from DHT actirity where hair growth is being induced.

According to embodiments, at least one growth factor for regrowing hair may be included in the composition as well. Such growth factors may be one or more of a hair follicle stimulator, a scalp blood supply circulation stimulator, and an agent promoting angiogenesis near hair follicles. According to embodiments, IGF-1 is the hair follicle stimulator. According to embodiments, a fibroblast growth factor, such as bFGF, is the scalp blood supply circulation stimulator. According to embodiments, VEGF is the blood supply circulation stimulator. Each of these growth factors may be added in a concentration of 1-10000 μg/ml, according to embodiments. According to embodiments, concentrations as low as 1-5 μg/ml for each growth factor are effective for effecting hair regrowth.

According to embodiments, at least one of KGF, TGF-β3, or TRX is also included in the composition.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising, in combination:
providing a composition having an agent that interferes with 5-α-reductase to prevent hair loss; and
providing at least one of:
a hair follicle stimulator;
a scalp blood supply circulation stimulator; and
an agent promoting angiogenesis near hair follicles; to stimulate hair growth.

2. The method of claim 1, wherein the hair follicle stimulator is insulin-like growth factor-1 (IGF-1).

3. The method of claim 1, wherein the scalp blood supply circulation stimulator is basic fibroblast growth factor (bFGF).

4. The method of claim 1, wherein the agent promoting hair follicle angiogenesis is vascular endothelial growth factor (VEGF).

5. The method of claim 1, wherein the hair follicle stimulator is IGF-1, the scalp blood supply circulation stimulator is bFGF, and the agent promoting hair follicle angiogenesis is VEGF.

6. The method of claim 1, wherein at least one of KGF, TGF-β3, and TRX is also provided to stimulate hair growth.

7. The method of claim 1, wherein the agent that interferes with 5-α-reductase is a peptide comprising copper.

8. The method of claim 6, wherein the peptide comprises at least three amino acids and copper (II).

9. The method of claim 8, wherein the peptide comprising copper has at least the sequence Gly-His-Lys-Cu(II).

10. The method of claim 8, wherein the peptide comprising copper has at least the sequence selected from the group consisting of: Cu(II)-His-Val-His, His-Ala-His-Cu(II), and Gly-His-Lys-Cu(II).

11. A composition comprising:
an agent that interferes with 5-α-reductase; and
at least one of;
a hair follicle stimulator;
a scalp blood supply circulation stimulator; and
an agent promoting angiogenesis near hair follicles.

12. The composition of claim 11, wherein the hair follicle stimulator is insulin-like growth factor-1 (IGF-1).

13. The composition of claim 11, wherein the scalp blood supply circulation stimulator is basic fibroblast growth factor (bFGF).

14. The composition of claim 11, wherein the agent promoting hair follicle angiogenesis is vascular endothelial growth factor (VEGF).

15. The composition of claim 11, wherein the agent: that interferes with 5-α-reductase is a peptide comprising copper.

16. The composition of claim 15, wherein the peptide comprising copper has at least the sequence Gly-His-Lys-Cu(II).

17. The method of claim 11, wherein at least one of KGF, TGF-β3, and TRX is also provided to stimulate hair growth.

18. A composition comprising:
an 5-α-reductage interfering agent, the agent having at least a peptide sequence comprising copper.

19. The composition, of claim 18, wherein the agent that interferes with 5-α-reductase is a peptide comprising copper.

20. The composition of claim 19, wherein peptide sequence comprising copper comprises at least Gly-His-Lys-Cu(II).
